(19) United States
(12) Patent Application Publication
(10) Pub. No.: US 2009/0304616 A1
(43) Pub. Date: Dec. 10, 2009

(54) TRANSPARENT ANTIPERSPIRANT GEL

(76) Inventors: BERNHARD BANOWSKI, Düsseldorf (DE); Melanie Schmidt, Krefeld (DE); Armin Wadle, Erkrath (DE)

Correspondence Address: Henkel Corporation, 10 Finderne Avenue, Bridgewater, NJ, 08807, US

(21) Appl. No.: 12/492,297

(22) Filed: Jun. 26, 2009

Related U.S. Application Data
(63) Continuation of application No. PCT/EP2007/063651, filed on Dec. 11, 2007.

(30) Foreign Application Priority Data
Dec. 29, 2006 (DE) 10 2006 062 564.1
Dec. 7, 2007 (DE) 10 2007 059 297.5

(51) Int. Cl. A61K 8/92

(52) U.S. Cl. 424/65

(57) ABSTRACT

The present application relates to clear antiperspirant gels in the form of a water-in-oil emulsion.

### CROSS-REFERENCE TO RELATED APPLICATIONS

The present application is a continuation of International Application No. PCT/EP2007/063651, filed 11 Dec. 2007, which claims the benefit of German Patent Application Nos. 10 2006 062 564.1, filed 29 Dec. 2006 and 10 2007 059 297.5, filed 7 Dec. 2007.

The present invention relates to clear, antiperspirant gels in the form of a water-in-oil emulsion.

Clear, antiperspirant and deodorant gel compositions are disclosed, for example, in U.S. Pat. No. 5,587,153. These compositions concern water-in-oil emulsions with a viscosity from about 50 Pa s to 250 Pa·s (50 000 to 250 000 cP), and preferably about 100 to 200 Pa·s (100 000 to 200 000 cP). The aqueous phase makes up about 75 to 90% of the composition and comprises a deodorant or antiperspirant active amount (e.g. about 3 to 25 wt. %) of an antiperspirant active substance. The oil phase makes up about 10 to 25 wt. % of the composition and comprises a silicone oil and a polyether-substituted silicone emulsifier. For optimal clarity, the refractive index of the oil phase and of the aqueous phase must be matched to within about 0.001 or better and preferably to within about 0.0004 of each other.

WO 96/06594 discloses transparent antiperspirant water-in-oil compositions that comprise silicone oils and/or volatile hydrocarbon oils and silicone-free emulsifiers.

EP 373424 discloses transparent antiperspirant water-in-oil compositions that comprise cyclotetrasiloxane and a mixture of silicone-free and silicone-containing emulsifiers.

WO 99/33440 A1 discloses antiperspirant compositions with reduced residue formation after application on the skin, comprising 5-80 wt. % cyclohexasiloxane, based on the total composition, 0-35 wt. % cyclotetrasiloxane and cyclopentasiloxane, based on the total fraction of Cyclomethicones in the composition, and a silicone elastomer.

The above described clear antiperspirant and deodorant gel compositions have the disadvantage of forming spots on clothing that comes into contact with the armpits of the user. Consequently, great efforts have been made to reduce or to eliminate staining of fabrics by changing the components of the compositions.

A further problem with the known gels is their high cyclomethicone content. With commercially available cyclomethicones, a distinction is made principally between cyclotetrasiloxane, cyclopentasiloxane and cyclohexasiloxane. Cyclotetrasiloxane, having an unusually high melting point of −11° C., can cause storage stability problems in the higher addition levels typically used for a water-in-oil emulsion gel. Cyclopentasiloxane is a relatively volatile oil component.

For this reason it is willingly employed in cosmetics, especially in antiperspirants, as it helps to solve the problem of staining clothing. Having said that, antiperspirants containing too high a content of volatile cyclopentasiloxane form white residues on the skin which adhere poorly to the skin and can slowly fall off, which is perceived as unpleasant by many consumers. Moreover, it has been determined that certain ingredients, such as silicone elastomers or oil-in-water emulsifiers, do not afford storage-stable transparent gel compositions.

An object of the present invention is to provide an antiperspirant or deodorant gel composition having reduced residue formation without a loss of efficacy or esthetic characteristics.

A further object of the present invention is to provide antiperspirant or deodorant gel compositions that comprise the lowest possible amounts of cyclotetrasiloxane and cyclopentasiloxane, preferably neither cyclotetrasiloxane nor cyclopentasiloxane.

A further object of the present invention is to provide clear, antiperspirant or deodorant gel compositions.

It was surprisingly found that the non-staining properties of the composition are substantially improved and that high storage stability in regard to the viscosity and phase separation under considerable variations in temperature can be achieved by using a high fraction of cyclohexasiloxane as the moderately volatile to volatile oil component.

The subject matter of the present application is a clear, transparent antiperspirant or deodorant gel composition in the form of a water-in-oil emulsion having a viscosity in the range 40 to 250 Pa·s at 21° C., comprising 70-90 wt. % of an aqueous phase, in which is dissolved 3% to 25 wt. % of at least one antiperspirant salt and at least one water-soluble polyhydric C₂-C₉ alkanol containing 2-6 hydroxyl groups and/or at least one water-soluble polyethylene glycol containing 3-20 ethylene oxide units and mixtures thereof, 10% to 30 wt. % of an oil phase, therein 5-25 wt. % cyclohexasiloxane, a total content of cyclotetrasiloxane and cyclopentasiloxane in an amount of no more than 5 wt. %, at least one polyether-substituted water-in-oil silicone emulsifier and 0% to 2 wt. % of a non-volatile oil.

Gel compositions according to the invention represent water-in-oil emulsions and preferably have a viscosity in the range 40 to 250 Pa·s (40 000 to 250 000 cP), preferably 50 to 150 Pa·s (50 000 to 150 000 cP) and particularly preferably 60 to 100 Pa s (60 000 to 100 000 cP) at 21° C.

The viscosity data refer to measurements with a rotation viscosimeter from the Brookfield Company using the spindle and the rpm as recommended by Brookfield in the handbook “More Solutions to Sticky Problems”: when using T-spindles and Helipath:

TABLE VIS-1
Brookfield Viscosimeter model LV and HA, upper limit of the optimal
viscosity range for the measurement with the given measurement
parameters in mPas (milliPascal × seconds)

| | Shear | | | | |
|---|---|---|---|---|---|
| | rate | Spindle | | | |
| Equipment | (rpm) | T-A | T-B | T-C | T-D |
| LVT | 0.3 | 66 600 | 133 000 | 333 000 | 666 000 |
| LVT | 0.6 | 33 300 | 66 600 | 166 00 | 333 000 |
| LVT | 1.5 | 13 300 | 26 600 | 133 000 | 333 000 |
| LVT | 3 | 6 660 | 13 300 | 33 300 | 66 600 |
| LVT | 6 | 3 330 | 6 660 | 16 600 | 33 300 |
| LVF | | | | | |
| LVT | 12 | 1 660 | 3 330 | 8 300 | 16 600 |
| LVF | | | | | |
| HAT | 0.5 | 800 000 | 1 600 000 | 4 000 000 | 8 000 000 |
| HAT | 1 | 400 000 | 800 000 | 2 000 000 | 4 000 000 |
| HAF | | | | | |
| HAF | 2 | 200 000 | 400 000 | 1 000 000 | 2 000 000 |
| HAT | 2.5 | 160 000 | 320 000 | 800 000 | 1 000 000 |
| HAT | 5 | 80 000 | 160 000 | 400 000 | 800 000 |
| HAF | | | | | |

TABLE VIS-2
Brookfield Viscosimeter model R, upper limit of the optimal viscosity
range for the measurement with the given measurement parameters
in mPas (milliPascal × seconds)

| | Shear rate | Spindle | | | |
|---|---|---|---|---|---|
| Equipment | (rpm) | T-A | T-B | T-C | T-D |
| RVT | 0.5 | 400 000 | 800 000 | 2 000 000 | 4 000 000 |
| RVT | 1.0 | 200 000 | 400 000 | 1 000 000 | 2 000 000 |
| RVT | 2.0 | 100 000 | 200 000 | 500 000 | 1 000 000 |
| RVF | 2.5 | 80 000 | 150 000 | 400 000 | 800 000 |
| RVF | 4 | 50 000 | 100 000 | 250 000 | 500 000 |
| RVT | 5 | 40 000 | 80 000 | 200 000 | 400 000 |

The above listed viscosities represent the upper limit for the optimal measurement range for each respective spindle-rpm combination. If two different measurement parameter combinations are possible for a viscosity range, then the spindle-rpm combination is selected that gives the higher scale division value. Moreover, the viscosity data refer to the composition 24 hours after manufacture and at a temperature of 21° C., wherein the measurements are carried out using a helipath.

The aqueous phase makes up about 70 to 90 wt. %, preferably 75 to 85 wt. % of the composition according to the invention and comprises therein a deodorant or antiperspirant active amount (3 to 25 wt. %) of a dissolved antiperspirant active substance. The oil phase makes up 10 to 30 wt. %, preferably 15 to 25 wt. % of the composition according to the invention. The total content of cyclotetrasiloxane and cyclopentasiloxane is no more than a maximum amount of 5 wt. %, preferably 1 to 3 wt. %, particularly preferably 0.5 to 1.5 wt. % and extremely preferably 0 wt. %, each based on the weight of the composition according to the invention.

In addition, gel compositions according to the invention comprise a polyether-substituted silicone emulsifier that is likewise included in the oil phase.

Antiperspirant and deodorant gel compositions according to the present invention are water-in-oil emulsions in which the aqueous phase makes up 70 to 90% of the composition. The antiperspirant salt is dissolved in the aqueous phase in order to achieve an antiperspirant or deodorant effect.

Gel compositions according to the invention further comprise at least one water-soluble polyhydric C₂-C₉ alkanol containing 2-6 hydroxyl groups and/or at least one water-soluble polyethylene glycol having 3-20 ethylene oxide units, as well as mixtures thereof. These components are preferably selected from 1,2-propylene glycol, 2-methyl-1,3-propanediol, glycerine, butylene glycols such as 1,2-butylene glycol, 1,3-butylene glycol and 1,4-butylene glycol, pentylene glycols such as 1,2-pentanediol and 1,5-pentanediol, hexanediols such as 1,6-hexanediol, hexanetriols such as 1,2,6-hexanetriol, 1,2-octanediol, 1,8-octanediol, dipropylene glycol, tripropylene glycol, diglycerine, triglycerine, erythritol, sorbitol xylitol, as well as mixtures of the cited substances. Suitable water-soluble polyethylene glycols are selected from PEG-3, PEG-4, PEG-6, PEG-7, PEG-8, PEG-9, PEG-10, PEG-12, PEG-14, PEG-16, PEG-18 and PEG-20, as well as mixtures thereof, wherein PEG-3 to PEG-8 are preferred. Sugars and certain sugar derivatives such as fructose, glucose, maltose, maltitol, mannitol, inositol, sucrose, trehalose and xylose are also inventively suitable.

Particularly preferred gel compositions according to the invention include those wherein the at least one water-soluble polyhydric C₂-C₉ alkanol containing 2-6 hydroxyl groups and/or at least one water-soluble polyethylene glycol having 3-20 ethylene oxide units is chosen from 1,2-propylene glycol, 2-methyl-1,3-propanediol, glycerine, butylene glycols such as 1,2-butylene glycol, 1,3-butylene glycol and 1,4-butylene glycol, pentylene glycols such as 1,2-pentanediol and 1,5-pentanediol, hexanediols such as 1,6-hexanediol, hexanetriols such as 1,2,6-hexanetriol, 1,2-octanediol, 1,8-octanediol, di-propylene glycol, tri-propylene glycol, di-glycerine, tri-glycerine, erythritol, sorbitol and xylitol, as well as mixtures of the substances named above.

The water-soluble polyhydric C₂-C₉ alkanols containing 2-6 hydroxyl groups or the water-soluble polyethylene glycol having 3-20 ethylene oxide units principally serve to adjust the refractive index of the aqueous phase (n_{D} (water)=1.33) to the value of the oil phase (n_{D} (oils)>1.38). The better the refractive indices of the oil phase and aqueous phase are aligned to one another, the higher is the transparency of the gel composition according to the invention. Preferably, in order to achieve a maximum clarity of the finished composition, the refractive indices of the oil phase and aqueous phase are aligned to one another to within ±0.0004, preferably to within ±0.0003. The gel composition preferably has a maximum cloudiness or lack of clarity of no more than 75 NTU (nephelometric turbidity units) and particularly preferably no more than 50 NTU at 21° C.

The water-soluble polyhydric C₂-C₉ alkanols containing 2-6 hydroxyl groups or the water-soluble polyethylene glycol having 3-20 ethylene oxide units also lend a skin-care hydrating action to the composition. Furthermore, they help to mask the residues of the gel composition on the skin.

Particularly preferred gel compositions according to the invention comprise the at least one water-soluble polyhydric C₂-C₉ alkanols containing 2-6 hydroxyl groups and/or the water-soluble polyethylene glycol having 3-20 ethylene oxide units in a total amount of 10-50 wt. %, preferably 15-30 wt. %, particularly preferably 18-25 wt. %, exceedingly preferably 20-23 wt. %, each based on weight of the total composition.

The fraction of water in the gel composition according to the invention is preferably 20-60 wt. %, particularly preferably 30-50 wt. %, exceedingly preferably 40-45 wt. %, each based on weight of the total composition.

Antiperspirant active substances that can be used in the gel compositions according to the invention include any conventional aluminum, zirconium and aluminum-zirconium salts, which are known to be suitable antiperspirant active substances. These salts include aluminum halides and aluminum hydroxy halides (e.g., aluminum chlorohydrate), as well as mixtures and complexes thereof with zirconyl oxy halides and zirconyl hydroxy halides (e.g., aluminum zirconium chlorohydrate).

Preferred antiperspirant active substances are chosen from water-soluble astringent inorganic and organic salts of aluminum, zirconium and zinc or any mixtures of these salts. Particularly preferred antiperspirant active substances are chosen from aluminum chlorohydrates, in particular aluminum chlorohydrates of the general formula [AI₂(OH)₅CI2-3H₂O]ₙ, which can be in non-activated or activated (depolymerized) form, furthermore aluminum sesquichlorohydrate, aluminum chlorohydrex-propylene glycol (PG) or -polyethylene glycol (PEG), aluminum sesquichlorohydrex-PG or -PEG, aluminum PG-dichlorohydrex or aluminum PEG-dichlorohydrex, aluminum hydroxide, additionally selected from the aluminum zirconium chlorohydrates, such as aluminum zirconium trichlorohydrate, aluminum zirconium tetrachlorohydrate, aluminum zirconium pentachlorohydrate, aluminum zirconium octachlorohydrate, the aluminum-zirconium chlorohydrate-glycine complexes such as aluminum zirconium trichlorohydrexglycine, aluminum zirconium tetrachlorohydrexglycine, aluminum zirconium pentachlorohydrexglycine, aluminum zirconium octachlorohydrexglycine, potassium aluminum sulfate (KAl(AI₂)₅CI 2-3H₂O, alum), aluminum undecylenoyl collagen amino acid, sodium aluminum lactate+aluminum sulfate, sodium aluminum chloro hydroxylactate, aluminum bromohydrate, aluminum chloride, the complexes of zinc and sodium salts, the complexes of lanthanum and cerium, the aluminum salts of lipoamino acids, aluminum sulfate, aluminum lactate, aluminum chlorohydroxy allantoinate, sodium aluminum chlorohydroxy lactate, zinc chloride, zinc sulfocarbolate, zinc sulfate and zirconium chlorohydrate. According to the invention, water-solubility is understood to mean a solubility of at least 5 wt. % at 20° C., i.e., that quantities of at least 5 g of the antiperspirant active substance are soluble in 95 g water at 20° C. In a particularly preferred embodiment, the composition comprises an astringent aluminum salt, especially aluminum chlorohydrate, which is commercially available, for example, in the form of an aqueous solution as Locron® L from Clariant, as Chlorhydrol® as well as in activated form as Reach® 501von from Reheis. An aluminum sesquichlorohydrate is offered by Reheis under the trade name Reach® 301, which is also particularly preferred. Also, the use of aluminum-zirconium tetrachlorohydrex-glycine complexes, which are commercialized, for example by Reheis under the trade name Rezal® 36G, can be particularly preferred according to the invention.

Further preferred aluminum salts are those with the general Formula AI₂(OH)₆₋ₐXₐ, wherein X is Cl, Br, I or NO₃ and “a” is 0.3 to 4 and preferably 1 to 2, such that the mole ratio of AI to X is in the range 1:1 to 2.1:1. As a rule, these salts comprise water of hydration, typically in amounts of 1 to 6 mole water per mole salt. The aluminum salt aluminum chlorohydrate (i.e. X=Cl) with “a”=1 is the most preferred, such that the mole ratio of aluminum to chlorine is 1.9:1 to 2.1:1.

Further preferred aluminum zirconium salts are mixtures or complexes of the above-described aluminum salts with zirconium salts of the formula ZrO(OH)_{2-pb}Y_{b}, wherein Y is Cl, Br, I, NO₃ or SO₄, “b” is 0.8 to 2 and “p” is the value of Y. As a rule, these salts comprise water of hydration, typically in amounts of 1 to 7 moles of water per mole of salt. The zirconium salt is preferably zirconyl hydroxychloride of the formula ZrO(OH)_{2-b}Cl_{b}, wherein “b” is 1 to 2 and preferably 1.2 to 1.9. The preferred aluminum zirconium salts have an Al:Zr ratio of 1.7 to 12.5 and most preferably 2 to 10, as well as a ratio of metal/(X+Y) of 0.73 to 2.1 and preferably 0.9 to 1.5. A preferred salt is aluminum zirconium chlorohydrate (i.e., X and Y are Cl) with an Al:Zr ratio of 2 to 10 and a metal:Cl ratio of 0.9-2.1, preferably 0.95-1.5, particularly preferably 1-1.3. For this reason the forms tri-, tetra-, penta- and octachlorohydrate should be included in the term “aluminum zirconium chlorohydrate”. The aluminum zirconium salt complexes can furthermore comprise a neutral amino acid, preferably glycine, typically with a gly:Zr ratio of approximately 1:1, i.e., 0.8-1.2, particularly preferably 1.

Gel compositions according to the invention comprise at least one antiperspirant active substance in an amount of 3-27 wt. %, preferably 5-22 wt. % and particularly 10-20 wt. %, each based on the total weight of the active substance (USP, US Pharmacopoeia) in the total composition. The amount in wt. % of antiperspirant salt(s) cited in the present patent application is calculated according to the method of the US Pharmacopoeia (USP), according to which bound water of crystallization and other ligands, e.g., glycine, are excluded. The antiperspirant salts are preferably employed in solubilized form, i.e., dissolved in a solvent, preferably dissolved in water, ethanol, propanol, isopropanol, water-soluble polyhydric C₂-C₉ alkanols containing 2-6 hydroxyl groups (see above) and mixtures of these solvents. Among these, preferably 1,2-propylene glycol, water-soluble polyethylene glycols having 3-20 ethylene oxide units (see above), and mixtures of these solvents, especially water/ethanol and water/1,2-propylene glycol. Antiperspirant salts are preferably employed as aqueous solutions, typically in a concentration of 30-50 wt. %. Such solutions are most preferably not prepared by redissolving spray dried salts, as spray dried salts comprise oxides that can cause turbidity in the finished composition.

Gel compositions according to the invention are in the form of a water-in-oil emulsion and comprise at least one polyether-substituted water-in-oil silicone emulsifier (W/O emulsifier) that stabilizes the emulsification of the aqueous phase into the oil phase.

An inventively particularly preferred group of water-in-oil silicone emulsifiers are the poly(C₂-C₃)-alkylene glycol-modified silicones, formerly called (INCI name) Dimethicone Copolyol, with the actual INCI names PEG-x Dimethicone (with x=2-20, preferably 3-17, particularly preferably 11-12), bis-PEG-y Dimethicone (with y=3-25, preferably 4-20), PEG/PPG a/b Dimethicone (wherein a and b independently of one another stand for numbers from 2-30, preferably 3-30 and particularly preferably 12-20, in particular 14-18), bis-PEG/PPG c/d Dimethicone (wherein c and d independently of one another stand for numbers from 10-25, preferably 14-20 and particularly preferably 14-16) and bis-PEG/PPG e/f PEG/PPG g/h Dimethicone (wherein e, f, g and h independently of one another stand for numbers from 10-20, preferably 14-18 und particularly preferably 16). PEG/PPG-18/18 Dimethicones are particularly preferred, which is available in a 1:9 mixture with Cyclomethicone as DC 3225 C or DC 5225 C, as well as bis-PEG/PPG-14/14 Dimethicone, which is commercially available in a mixture with cyclopentasiloxane in the weight ratio 85 (emulsifier):15 (cyclopentasiloxane) as Abil EM 97 (Goldschmidt).

It was surprisingly found that the water-in-oil silicone emulsifier bis-PEG/PPG-14/14 Dimethicone enables a gentle and energy-saving emulsion preparation at relatively low shear rates or low shearing energy input. On the other hand, emulsion preparations with PEG/PPG-18/18 Dimethicone/Cyclomethicone solutions requires a high shearing energy input. Accordingly, the water-in-oil silicone emulsifier bis-PEG/PPG-14/14 Dimethicone is particularly preferred according to the invention.

Further extremely preferred W/O emulsifiers according to the invention are poly(C₂-C₃) alkylene glycol-modified silicones that are hydrophobically modified with C₄-C₁₈ alkyl groups. Surprising advantages in the preparation and processing also resulted here. Particularly preferred W/O silicone emulsifiers are cetyl PEG/PPG-10/1 Dimethicone (formerly: Cetyl Dimethicone Copolyol, available as Abil EM 90), Lauryl Dimethicone Copolyol, PEG-8 Cetyl Dimethicone, as well as Alkyl Methicone Copolyols and Alkyl Dimethicone Ethoxy Glucoside.

Particularly preferred gel compositions according to the invention comprise at least one organic W/O silicone emulsifier in an amount of 0.1 to 10 wt. %, preferably 0.5 to 5 wt. %, particularly preferably 1-3 wt. % and extremely preferably 1.2-2,1 wt. %, each based on the total weight of the composition according to the invention.

Particularly preferred gel compositions according to the invention comprise as the sole W/O silicone emulsifier bis-PEG/PPG-14/14 Dimethicone in an amount of 2-3.5 wt. %, preferably 2.1-3 wt. %, based on the total weight of the composition according to the invention.

Further particularly preferred gel compositions according to the invention comprise as the sole W/O silicone emulsifier Cetyl PEG/PPG-10/1 Dimethicone in an amount of 1-3 wt. %, based on the total weight of the composition according to the invention.

Further particularly preferred gel compositions according to the invention comprise as the sole W/O silicone emulsifier Lauryl Dimethicone Copolyol in an amount of 1-3 wt. %, based on the total weight of the composition according to the invention.

Gel compositions according to the invention preferably comprise at least 5 wt. % ethanol, based on total weight of the composition. An ethanol content of 8-20 wt. % is preferred and 10-15 wt. % ethanol is extremely preferred. It was surprisingly found that incorporating ethanol in the W/O emulsion makes the ethanol considerably more skin compatible. This was especially observed in W/O emulsions that comprise bis-PEG/PPG-14/14 Dimethicone or Cetyl PEG/PPG-10/1 Dimethicone or Lauryl Dimethicone Copolyol as the sole silicone emulsifier. Specifically with bis-PEG/PPG-14/14 Dimethicone, it was also surprisingly observed that the freshening effect, generated by the ethanol content of the W/O emulsion according to the invention, corresponds to or even surpasses that of the conventional ethanolic solution, in fact in spite of the higher water content.

In a preferred embodiment, gel compositions according to the invention are exempt of silicone elastomers.

According to the invention, silicone elastomers are understood to mean those silicone elastomers that are obtained by crosslinking an organopolysiloxane that comprises in each molecule at least 2 C₂-C₁₀ alkenyl groups having terminal double bonds, with an organopolysiloxane that has in each molecule at least two silicon-bonded hydrogen atoms. The organopolysiloxane containing at least 2 C₂-C₁₀ alkenyl groups with a terminal double bond in the molecule is selected from methylvinylsiloxanes, methylvinylsiloxane-dimethylsiloxane copolymers, dimethylpolysiloxanes with dimethylvinylsiloxy end groups, dimethylsiloxane-methylphenylsiloxane copolymers with dimethylvinylsiloxy end groups, dimethylsiloxane-diphenylsiloxane-methylvinylsiloxane copolymers with dimethylvinylsiloxy end groups, dimethylsiloxane-methylvinylsiloxane copolymers with trimethylsiloxy end groups, dimethylsiloxane-methylphenylsiloxane-methylvinylsiloxane copolymers with trimethylsiloxy end groups, methyl-(3,3,3-trifluoropropyl)-polysiloxanes with dimethylvinylsiloxy end groups and dimethylsiloxane-methyl-(3,3,3-trifluoropropyl)-siloxane copolymers with dimethylvinylsiloxy end groups.

Crosslinking organopolysiloxanes with two silicon-bonded hydrogen atoms are chosen from methylhydrogenpolysiloxanes with trimethylsiloxy end groups, dimethylsiloxane-methylhydrogensiloxane copolymers with trimethylsiloxy end groups and cyclic dimethylsiloxane-methylhydrogen-siloxane copolymers.

Non-emulsifying silicone elastomers are commercially available, for example, from Dow Corning as the products DC 9040, DC 9041 or DC 9509, from General Electric, e.g., as the commercial products SFE 839 and GE 1229, from Shin-Etsu as the commercial products KSG-15, KSG-16 or KSG-18 as well as from Grant Industries as the products from the Gransil® series, such as e.g., Gransil®RPS Gel (INCI-name Cyclopentasiloxane and Polysilicone-11) or Gransil®GCM-4 (INCI-name Cyclotetrasiloxane and Polysilicone-11).

Emulsifying silicone elastomers comprise polyoxyethylene groups and/or polyoxypropylene groups as the functional groups on the polysiloxane backbone. These groups can be terminal groups and/or can be located as side chains on the polysiloxane chain. Emulsifying silicone elastomers are commercially available, for example, from Shin-Etsu as the products KSG-21, KSG-31, KSG-31X, KSG-32 or from Dow Corning as the commercial product DC-9011.

In a further preferred embodiment, gel compositions according to the invention are exempt from oil-in-water emulsions, in particular exempt from oil-in-water emulsifiers with an HLB value of 12 and above.

The oil phase makes up 10 to 30 wt. %, preferably 15-25 wt. %, particularly preferably 17-22 wt. %, each based on total weight of the gel composition according to the invention. The oil phase represents the external phase and lends the composition a particularly soft, pleasant skin feel and, in addition to the antiperspirant action, a cosmetic care action.

The cyclohexasiloxane content is 5 to 25 wt. %, preferably 8 to 20 wt. % and particularly preferably 10 to 15 wt. %, based on total weight of the composition according to the invention.

The total content of cyclotetrasiloxane and cyclopentasiloxane is no more than a maximum of 5 wt. %, preferably 1 to 3 wt. %, particularly preferably 0.5 to 1.5 wt. %, extremely preferably 0 wt. %, each based on total weight of the gel composition according to the invention.

It was determined that the presence of significant quantities of non-volatile oils, i.e., 2 wt. % or more, based on total weight of the gel composition according to the invention, causes the compositions to stain items of clothing. These types of non-volatile oils incorporate non-volatile silicones, such as for example polydimethylsiloxanes (Dimethicones) with a kinematic viscosity (25° C.) of 1×10⁻⁵ m²/s (10 cSt) and above, as well as other organic oils as the emollient, such as for example the esters of linear or branched saturated or unsaturated fatty alcohols containing 2-30 carbon atoms with linear or branched saturated or unsaturated fatty acids containing 2-30 carbon atoms, which can be hydroxylated, for example octyl isononanoate. These oils are normally included in the composition in order to enable it to impart suppleness to the skin and to prevent the final product from adhering or being sticky. Up to now it was not possible to reduce the non-volatile oil components of the gel component without increasing the disadvantageous effect on the esthetic characteristics of the product.

Preferred gel compositions according to the invention are exempt from hydrocarbon oils that can comprise oxygen atoms, such as the abovementioned ester oils, and which are not fragrances.

Further preferred gel compositions according to the invention are exempt from polydimethylsiloxanes (Dimethicones) with a kinematic viscosity (25° C.) of 1×10⁻⁵ m²/s (10 cSt) and above.

Preferred gel compositions according to the invention comprise at least one volatile linear silicone with a kinematic viscosity (25° C.) below 1×10⁻⁵ m²/s (10 cSt).

The volatile linear silicone preferably serves to replace a part or the whole of the non-volatile oil components. This volatile linear silicone is a polydimethylsiloxane or Dimethicone that has a comparatively low relative molecular weight, a relatively low viscosity and a significant vapor pressure at 25° C. (i.e. one gram of the liquid deposited on filter paper no. 1, leaves essentially no visible residue after 30 minutes at room temperature). It moreover typically has a boiling point below 250° C. The volatile silicone (or the volatile Dimethicone) are represented by the formula (CH₃)₃SiO(Si(CH₃)₂O)ₙSi(CH₃)₃, in which n is a whole number from zero to 6 and is preferably 1 to 4. In order to provide an alkylmethylsiloxane, one of the methyl groups of the above formula can be replaced by an alkyl group (e.g., containing 2 to 10 carbon atoms). Such material includes, for example, DC 2-1731 (Dow Corning), which is 3-hexylheptamethyltrisiloxane (viscosity 1×10⁻⁶ m²/s (1.0 cSt)). Although a pure silicone polymer can be employed, the volatile linear silicone is usually a mixture of silicone polymers of the abovementioned Formula. The volatile linear silicone will have a viscosity of less than 5×10⁻⁶ m²/s (5 cSt or less than about 5 cP) and preferably between 0.6×10⁻⁶ and 3×10⁻⁶ m²/s (0.6 and 3.0 cSt) and more preferably between 1×10⁻⁶ and 2×10⁻⁶ m²/s (1.0 and 2.0 cSt). For silicones with a specific gravity at 25° C. in the range 0.75 to 0.92, the above cited viscosity ranges become 0.0005 to 0.0028 Pa·s (0.5 to 2.8 cP) and preferably about 0.0008 to 0.0018 Pa·s (0.8 to 1.8 cP). Suitable volatile linear silicones include Dimethicones with 6.5×10⁻⁵ m²/s (0.65 cSt) (hexamethyldisiloxane), Dimethicone 10⁻⁶ m²/s (1.0 cSt) (octamethyltrisiloxane), Dimethicone 1.5×10⁻⁶ m²/s (1.5 cSt), Dimethicone 2.0×10⁻⁶ m²/s (2.0 cSt) (dodecamethylpentasiloxane), DC2-1184 and DC2-1731, which are all available from Dow Corning. DC2-1184, which has a viscosity of about 1.7×10⁻⁶ m²/s (1.7 cSt) and a mean relative molecular weight of about 320 (i.e., n is about 1 to 3 in the above mentioned formula), is preferred.

The amount of volatile linear silicone compounded into the composition depends on the properties of the specific volatile linear silicone employed, as well as other oil components present in the composition. This means that the amount of volatile linear silicone and the amount of moderately volatile to volatile cyclohexasiloxane can be balanced out so as to achieve the desired balance of freedom from stains and absence of stickiness or the suppleness of the skin. The volatile linear silicone is preferably employed in a quantity of 2-10 wt. % and particularly preferably 3% to 8 wt. %, based on total weight of the gel composition according to the invention.

Gel compositions according to the invention preferably further comprise at least one fragrance component. Suitable perfume oils or fragrances that can be used include individual odoriferous compounds, for example synthetic products of the ester, ether, aldehyde, ketone, alcohol and hydrocarbon type. Odoriferous compounds of the ester type are, for example phenoxyethyl isobutyrate, benzyl acetate, p-tert.-butylcyclohexyl acetate, dimethylbenzyl carbinyl acetate, linalyl acetate, phenylethyl acetate, linalyl benzoate, ethylmethylphenyl glycinate, benzyl formate, allylcyclohexyl propionate, styrallyl propionate and benzyl salicylate. Ethers include, for example, benzyl ethyl ether; the aldehydes include, for example, the linear alkanals containing 8 to 18 carbon atoms, citral, citronellal, citronellyloxyacetaldehyde, cyclamen aldehyde, hydroxycitronellal, lilial and bourgeonal. Ketones include, for example, the ionones, α-isomethyl ionone and methyl cedryl ketone. Alcohols include anethol, citronellol, eugenol, geraniol, linalool, phenylethyl alcohol and terpineol and the hydrocarbons include, above all, the terpenes and balms. However, mixtures of various odoriferous substances, which together produce an attractive fragrant note, are preferably used. Suitable perfume oils can also contain natural mixtures of odoriferous substances obtainable from vegetal or animal sources, for example, pine, citrus, jasmine, lilac, rose or ylang-ylang oil. The ethereal oils of lower volatility that are mostly used as aroma components are also suitable as perfume oils, e.g., oil of sage, chamomile oil, clove oil, melissa oil, mint oil, cinnamon leaf oil, lime blossom oil, juniper berry oil, vetivert oil, olibanum oil, galbanum oil and laudanum oil.

The fragrance component(s) is/are preferably comprised in amounts of 0.01-4 wt. %, preferably 0.5-2 wt. %, each based on total weight of the gel composition according to the invention.

Gel compositions according to the invention can advantageously further comprise at least one skin-cooling active substance. Inventively suitable skin-cooling active substances include, for example, menthol, isopulegol as well menthol derivate, e.g., menthyl lactate, menthyl glycolate, menthyl pyrrolidone carboxylic acid, menthyl methyl ether, menthoxypropanediol, menthone glycerine acetal (9-methyl-6-(1-methylethyl)-1,4-dioxaspiro(4,5)decane-2-methanol), monomenthyl succinate and 2-hydroxymethyl-3,5,5-trimethylcyclohexanol. Preferred skin-cooling active substances include menthol, isopulegol, menthyl lactate, menthoxypropanediol and menthylpyrrolidone carboxylic acid as well as mixtures of these substances, in particular mixtures of menthol and menthyl lactate, menthol, menthol glycolate and menthyl lactate, menthol and menthoxypropanediol or menthol and isopulegol.

The inventive gel compositions preferably comprise at least one skin-cooling active substance in amounts of 0.01-1 wt. %, preferably 0.02-0.5 wt. % and particularly preferably 0.05-0.2 wt. %, each based on total weight of the gel composition.

Although compositions according to the present invention containing the above-described ingredients can be prepared and used, it may also be desirable to add other optional components in order to attain the aimed esthetic and other effects. For example, it may be desirable to add antimicrobials or deodorants, such as for example Triclosan, preservatives and/or complexants.

The invention is further described with the help of the following examples, which are only for the purpose of illustration. All fractions and weight data are based on total weight of the gel composition.

| | 1 | 2 |
|---|---|---|
| ALUMINUM ZIRCONIUM TETRA- | 6 | 23.5 |
| CHLOROHYDREX GLY | | |
| (50% conc. aqueous solution) | | |
| 1,2-Propylene glycol | 34.7 | 8.7 |
| Ethanol | — | 10 |
| DC 2-1184 (Dow Corning) | 3 | 4.8 |
| Cyclohexasiloxane, PEG/PPG-18/18 | 9 | 8.1 |
| Dimethicone | | |
| DC 246 | 7 | 5 |
| Phenoxyethanol | 1.15 | — |
| Perfume | 0.4 | 0.2 |
| Water | ad 100 | ad 100 |

DC2-1184 (Dow Corning): Mixture of linear polydimethylsiloxanes (mean molecular weight approx. 320 g/mol; viscosity approx. 1.7 cSt)

DC 0.65 cSt, 200 Fluid (Dow Corning): Hexamethylenedisiloxane (mean molecular weight approx. 162 g/mol; viscosity approx. 0.65 cSt)

DC 246 (Dow Corning): at least 95 wt. % cyclohexasiloxane, max. 5 wt. % cyclopentasiloxane

| | No. 3 | No. 4 | No. 5 | No. 6 | No. 7 |
|---|---|---|---|---|---|
| Abil EM 97 | 3.0 | 3.5 | 2.5 | 3.2 | 3.0 |
| Dow Corning 246 | 14.2 | 14.2 | 14.2 | 14.2 | 14.2 |
| Ethanol (96% | 10 | 5.3 | 10 | 8 | 10 |
| DEP denatured) | | | | | |
| Perfume/Fragrance | 0.6 | 0.6 | 1.0 | 1.3 | 1.0 |
| Aluminum | 20 | 20 | 22 | 18 | 19 |
| hydroxychloride | | | | | |
| (active substance, | | | | | |
| USP) | | | | | |
| 1,2-Propylene glycol | 21.5 | 18 | 23 | 18 | 20 |
| Water | ad 100 | ad 100 | ad 100 | ad 100 | ad 100 |

The abovementioned compositions were manufactured in the following way. The components of the aqueous phase and the components of the oil phase were each mixed in separate containers and optionally filtered, and the refractive indices of each phase were measured. The refractive index of the aqueous phase was adjusted to match the refractive index of the oil phase to within 0.0004, in that, as needed, water or a water-soluble polyhydric C₂-C₈ alkanol containing 2-6 hydroxyl groups and/or at least one water-soluble polyethylene glycol having 3-20 ethylene oxide units, preferably 1,2-propylene glycol, were added. The aqueous phase was then slowly added to the oil phase at room temperature (e.g. 18° C.) under sufficient mixing to produce a clear emulsion, optionally with minimal ventilation. This emulsion was then sheared, producing a clear gel with a viscosity of approx. 40 to 250 Pas (40 000 to 250 000 cP).

We claim:

1. Antiperspirant or deodorant gel composition comprising:
a) 70-90 wt. % of an aqueous phase in which is dissolved 3% to 25 wt. % of at least one antiperspirant salt, and
at least one water-soluble polyhydric C₂-C₉ alkanol containing 2-6 hydroxyl groups and/or at least one water-soluble polyethylene glycol containing 3-20 ethylene oxide units and mixtures thereof,
b) 10% to 30 wt. % of an oil phase having therein 5-25 wt. % cyclohexasiloxane,
no more than 5 wt. % of cyclotetrasiloxane and cyclopentasiloxane,
at least one polyether-substituted water-in-oil silicone emulsifier and
0% to 2 wt. % of a non-volatile oil,
wherein all weight percentages are based on total weight of the gel composition,
wherein the gel composition is in the form of a water-in-oil emulsion having a viscosity in the range of 40 to 250 Pa·s at 21° C., and
wherein the gel composition is clear.

2. Gel composition according to claim 1, wherein the viscosity is in the range of 50 to 150 Pa·s at 21° C.

3. Gel composition according to claim 1 comprising the at least one water-soluble polyhydric C₂-C₉ alkanol containing 2-6 hydroxyl groups and/or the at least one water-soluble polyethylene glycol having 3-20 ethylene oxide units in a total amount of 10-50 wt. %, based on total weight of the gel composition.

4. Gel composition according to claim 1 further comprising at least 5 wt. % ethanol based on total weight of the gel composition.

5. Gel composition according to claim 1, wherein the polyether-substituted water-in-oil silicone emulsifier is chosen from bis-PEG/PPG-14/14 Dimethicone, Cetyl PEG/PPG-10/1 Dimethicone and Lauryl Dimethicone Copolyol.

6. Gel composition according to claim 1 further comprising at least one W/O silicone emulsifier in an amount of 0.1 to 10 wt. %, based on total weight of the gel composition.

7. Gel composition according to claim 1 further comprising bis-PEG/PPG-14/14 Dimethicone as a water-in-oil silicone emulsifier, wherein the bis-PEG/PPG-14/14 Dimethicone is the only water-in-oil silicone emulsifier, and at least 5 wt. % ethanol, based on total weight of the gel composition.

8. Gel composition according to claim 1 further comprising at least one volatile linear silicone.

9. Gel composition according to claim 8, wherein the at least one volatile linear silicone is present in an amount of 2 to 10 wt. % based on total weight of the gel composition.

10. Gel composition according to claim 1, wherein the composition has a maximum lack of clarity of 75 NTU (Nephelometric Turbidity Units) at 21° C.

11. Gel composition according to claim 1 comprising at least 20-60 wt. % water, based on total weight of the gel composition.

12. Gel composition according to claim 1 wherein the composition is exempt from silicone elastomers.

13. Gel composition according to claim 1, wherein the composition is exempt from oil-in-water emulsifiers.

14. Process for preparing an antiperspirant or deodorant gel composition comprising:
separately mixing the components of the aqueous phase and the components of the oil phase,
measuring the refractive indices of each phase,
adjusting the refractive index of the aqueous phase to match the refractive index of the oil phase to within 0.0004,
adding the aqueous phase to the oil phase under sufficient mixing, and
shearing the resultant water-in-oil emulsion to produce an antiperspirant or deodorant gel composition.

15. Process according to claim 14 wherein the refractive index of the aqueous phase is adjusted by adding water, a water-soluble polyhydric C₂-C₉ alkanol containing 2-6 hydroxyl groups, and/or at least one water-soluble polyethylene glycol having 3-20 ethylene oxide units, preferably 1,2-propylene glycol.

16. Process according to claim 14 wherein the at least one water-soluble polyethylene glycol having 3-20 ethylene oxide units is at least 1,2-propylene glycol.

17. Process according to claim 14 wherein the aqueous phase is added to the oil phase at room temperature.

18. Process according to claim 14 wherein the gel composition has a maximum cloudiness of 75 NTU (Nephelometric Turbidity Units) at 21° C.
